(19) United States
(12) Plant Patent Application Publication
(10) Pub. No.: US 2002/0184681 P1
(43) Pub. Date: Dec. 5, 2002

(54) Chrysanthemum plant named 'Gedi One Ylcel'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,859

(22) Filed: Jun. 4, 2001

(51) Int. Cl. A01H 5/00

(52) U.S. Cl. PLT/289

(57) ABSTRACT

A new and distinct Chrysanthemum plant variety characterized by a decorative type inflorescence, consistent flowering response to short days, blooming consistently after 49 days of short day length, free branching habit, light yellow ray florets, resistant to fading with age, and very uniform round growth habit.

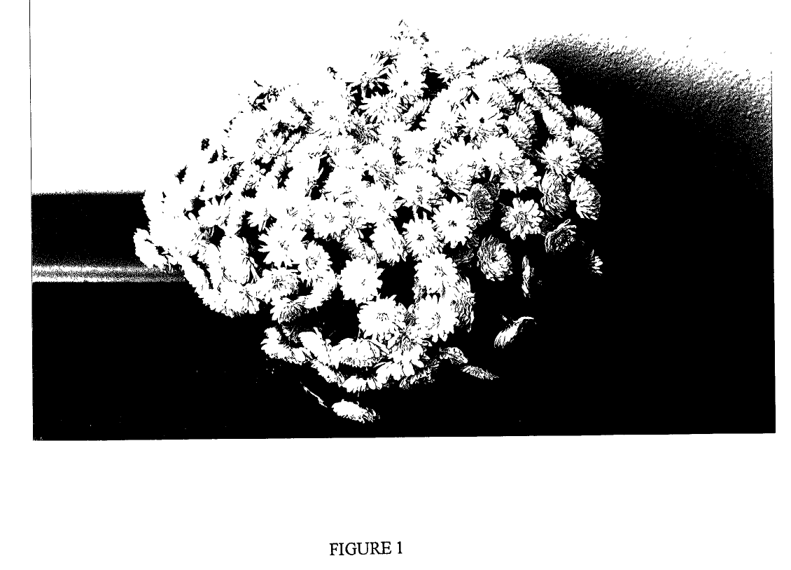

### BACKGROUND OF THE INVENTION

The present invention relates to a new and distinct cultivar of Chrysanthemum plant, botanically known as Dendranthema morifolium and herein after referred to by the cultivar name ‘GEDI ONE YLCEL.’

This new cultivar originated from a naturally occurring mutation on a single branch of a Chrysanthemum plant, cultivar ‘Celino’ (unpatented in the United States). The new cultivar was discovered in the inventor's field in Belgium in October 1998.

Asexual reproduction of the new cultivar by apical tip cutting and meristem tissue culture was performed in Oxnard, Cailf. and has shown that the unique features of this new cultivar are stable and reproduced true to type on successive generations.

### SUMMARY IF THE INVENTION

The cultivar ‘GEDI ONE YLCEL’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE YLCEL.’ These characteristics in combination distinguish ‘GEDI ONE YLCEL’ as a new and distinct Chrysanthemum:

1. Decorative type inflorescence;

2. Consistent flowering response to short days, blooming consistently after 49 days of short day length;

3. Free branching habit;

4. Light yellow ray florets, resistant to fading with age; and

5. Very uniform round growth habit.

Plants of the new cultivar are similar to plants of the parent cultivar, ‘Celino,’ in most horticultural characteristics, however plants of the new cultivar differed from plants of the cultivar ‘Celino’ in ray floret color.

The new cultivar can be compared to the Chrysanthemum cultivar ‘Dark Veria’ (disclosed in U.S. Plant Pat. No. 10,215). However plants of the new Chrysanthemum differed from the plants of the cultivar ‘Dark Veria’ in the following characteristics:

1. The new cultivar naturally blooms approximately 1 week later;

2. The new cultivar is taller and wider;

3. The new cultivar has a more round plant shape and greater branching;

4. The new cultivar has more blooms per flowering branch; and

5. The new cultivar has lighter yellow ray florets.

### BRIEF DESCRIPTION OF THE PHOTOGRAPHS

The photograph shows a plant of ‘GEDI ONE YLCEL’ grown in a 6-inch container. A single cutting was used in the container.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Cailf. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE YLCEL’

Commercial classification: Garden-type Chrysanthemum

Parentage: Naturally occurring mutation of a single branch on Chrysanthemum plant cultivar ‘Celino’ (unpatented in the United States)

### Propagation

Time to Rooting: 7 to 14 days at approximately 21° C.

Rooting habit: Fine, fibrous

### Plant

Growth Habit: Mounding herbaceous perennial

Height: Approximately 18.5 cm

Growth rate: Moderate

Branching Characteristics: Free Branching

Length of Lateral Branches: Approximately 15 cm

Number of leaves per lateral branch: Approximately 18

### Foliage

Leaf:

Arrangement.—Alternate

Average length.—Approximately 5.5 cm

Average width.—Approximately 4.0 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 147A young foliage under side: Near 147A mature foliage upper side: Near 147B mature foliage under side: Near 147B venation upper side: Near 147C venation under side: Near 147C

Petiole:

Average Length.—Approximately 1.5 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last week in September

Number of inflorescences per stem.—Approximately 10

Inflorescence diameter.—Approximately 3.8 cm

Inflorescence depth.—Approximately 1.7 cm

Inflorescence longevity on plant.—Approximately 26-29 days

Persistence.—Persistent

Ray Florets:

Texture.—Shiny

Average Number per flower.—Approximately 150

Shape.—Oblanceolate

Margin.—Entire

Apex.—Obtuse

Length.—Approximately 1.4 cm

Width.—Approximately 0.4 cm

Color.—upper surface at first opening: Near 5B upper surface at maturity: Near 154D upper surface at fading: Near 1D under surface at first opening: Near 5B under surface at maturity: Near 154D under surface at fading: Near 1D

Disc florets:

Appearance.—Shiny

Texture.—Smooth

Average number per flower.—Approximately 24

Shape.—Cylindric

Apex.—Obtuse

Average Length.—Approximately 0.6 cm

Average Width.—Approximately 0.2 cm

Color.—at first opening: Near 137B at maturity: Near 137B at fading: Near 137C

Peduncle:

Length.—at terminal end (shortest): Approximately 3.5 cm at lateral end (longest): Approximately 7 cm

Strength.—Moderate

Color.—Near 137D

Habit.—Erect

Inflorescence bud:

Length.—Approximately 0.6 cm

Diameter.—Approximately 0.5 cm

Form.—Globular

Color.—Near 2A

Sepals:

Appearance.—Matte

Texture.—Lightly pubescent

Number.—Approximately 22

Shape.—Ovate

Margin.—Entire

Apex.—Acute

Color.—upper side: Near 137C under side: Near 137B

### Reproductive Organs

Ray Florets:

Pistillate.—Number of pistils per flower: 1 Stigma shape: 2 branched Stigma color: Near 12A Stigma length: Approximately 0.1 cm Style color: Near 154C Style length: Approximately 0.2 cm

Disc Florets:

Pistillate.—Number of pistils per flower: 1 Stigma shape: Cylindric Style length: Approximately 0.5 cm

Staminate.—Number of stamens per flower: Approximately 5 Anther shape: Tubular Anther color: Near 12A Pollen color: Near 12A

### Other Characteristics

Disease Resistance: Neither resistance nor susceptibility observed

What is claimed is:

1. A new and distinct variety of Chrysanthemum plant named ‘GEDI ONE YLCEL’ as herein illustrated and described.
